(19) United States
(12) Patent Application Publication
(10) Pub. No.: US 2026/0077118 A1
(43) Pub. Date: Mar. 19, 2026

(54) WEARABLE FLUID DELIVERY SYSTEM WITH PRESSURE MEASUREMENT SYSTEM EXTERNALIZED FROM FLUID PATH BETWEEN RESERVOIR AND OUTLET TO PATIENT

(71) Applicant: Becton, Dickinson and Company, Franklin Lakes, NJ (US)

(72) Inventors: Clémentine Le Loc'h, Meylan (FR); Danielle Aboud, Clonskeagh (IE); Olivier Albenge, Vif (FR); Nicolas Besson, Treffort (FR)

(73) Assignee: Becton, Dickinson and Company, Franklin Lakes, NJ (US)

(21) Appl. No.: 18/884,259

(22) Filed: Sep. 13, 2024

(51) Int. Cl. A61M 5/142; A61M 5/168

(52) U.S. Cl. CPC A61M 5/14228; A61M 5/16854; A61M 5/16886; A61M 2005/16863; A61M 2205/0216; A61M 2205/025; A61M 2205/106; A61M 2205/3306; A61M 2205/3327; A61M 2205/3334; A61M 2205/3375

(57) ABSTRACT

A wearable fluid delivery device (FDD) has a fluidic module and an electromechanical module. The fluidic module has a fluid path from a fluid reservoir to an outlet to a patient that is separate from the electromechanical module and its components that control the delivery of the fluid to the patient. A pump that can be disposed in the fluidic module, or in the electromechanical module and external to the fluid path. The FDD can have a releasably coupled first housing and second housing that enclose a respective one of the fluidic module and the electromechanical module. The first and second housings when connected have respective ones of aligned sensor opening and sensor access opening to create a sensor access area and permit a sensor to detect a pressure in the fluid path and thereby provide a pressure sensing system external to the fluid path.

### BACKGROUND

### Field

The present disclosure relates generally to fluid delivery devices for delivering a medicament to a patient. The present disclosure particularly relates to a fluid delivery device having a fluidics module with a fluid path from a fluid reservoir to an outlet to the patient that is separate from an electromechanical module with components to control the delivery of the fluid from the fluid reservoir to the patient. Further, the present disclosure relates to the fluid delivery device having a pressure sensing system that is external to the fluid path.

### Description of Related Art

Bolus and/or infusion pump therapy generally requires an infusion cannula, typically in the form of an infusion needle and/or a flexible catheter, that pierces the patient's skin and through which, infusion of a medicament takes place. Infusion pump therapy offers the advantages of continuous infusion, precision dosing, and programmable delivery schedules.

To facilitate drug or medicament delivery therapy, there are generally two types of pumps, namely, conventional pumps and patch pumps. Conventional pumps require the use of a disposable component, typically referred to as an infusion set, tubing set or pump set, which conveys a medicament from a reservoir within the pump into the skin of the user. The infusion set consists of a pump connector, a length of tubing, and a hub or base from which a cannula in the form of a hollow metal infusion needle or flexible plastic catheter extends. The base typically has an adhesive that retains the base on the skin surface of a user during use of the pump. The cannula can be inserted onto the skin manually or with the aid of a manual or automatic insertion device. The insertion device may be a separate unit required by the user.

Unlike a conventional infusion pump and infusion set combination, a patch pump is an integrated device that combines most or all of the fluidic components, including the fluid reservoir, pumping mechanism and mechanism for inserting the cannula, in a single housing that is adhesively attached to an infusion site on the patient's skin, and generally does not require the use of a separate infusion or tubing set. A patch pump containing a medicament adheres to the patient's skin and delivers the medicament over a period of time or at a selected time via an integrated subcutaneous cannula. Some patch pumps may wirelessly communicate with a separate controller device, while others are completely self-contained. Such devices can be replaced on a frequent basis, such as every three days, when the medicament reservoir is exhausted or a component malfunctions, or when complications may otherwise occur, such as restriction in the cannula or the infusion site or other occlusion. Measurement of fluid pressure along a fluid path in the patch pump can be desirable to determine component malfunctions such as restriction in the cannula or the infusion site or other occlusion condition along the fluid path between the reservoir and the cannula.

### SUMMARY

In accordance with illustrative embodiments of the present disclosure, a wearable fluid delivery device (FDD) comprises two parts; that is, a fluidic module and an electromechanical module. The fluidics module has a fluid path from a fluid reservoir to an outlet to the patient that is separate from the electromechanical module and its components that control the delivery of the fluid from the fluid reservoir to the patient. The wearable FDD can have a pressure sensing system that is external to the fluid path from the fluid reservoir to the outlet to the patient. The wearable FDD realizes a number of advantages described below.

It is an aspect of illustrative embodiments to provide a fluid delivery device comprising: a first housing comprising a fluidics module and a sensor access opening, the fluidics module comprising a reservoir of fluid and a fluid path that fluidically connects the reservoir to an outlet configured to output the fluid from the fluid path to a patient, the fluid path comprising a sensor access area covered by a membrane configured to be sealed relative to the fluid path to retain the fluid in the fluid path; and a second housing separate from the first housing and comprising an electromechanical module and a sensor opening, the sensor opening and the sensor access opening being at least partially aligned when the second housing is connected to first housing, the electromechanical module comprising a sensor and electronics configured to process data output from the sensor, the sensor comprising a sensor contact surface disposed at the sensor opening of the second housing and configured to cooperate with at least part of the membrane in the sensor access area in the first housing to measure pressure of the fluid in the fluid path.

In accordance with aspects of illustrative embodiments, the sensor access area is formed by a casing fluidically connected to the fluid path and having side walls that define the sensor access area, and the membrane is a rigid material and is operable to be displaced along the side walls in response to pressure from the fluid contacting a fluid path facing side of the membrane, the sensor having a pressure sensing mechanism configured to detect displacement of the sensor contact surface when the sensor contact surface is in contact with the membrane and moves due to displacement of the membrane.

In accordance with aspects of illustrative embodiments, the membrane is a flexible material and is deformable in response to pressure from the fluid contacting a fluid path facing side of the membrane, the sensor having a pressure sensing mechanism configured to detect movement of the sensor contact surface when the sensor contact surface is in contact with the membrane and moves due to deformation of the membrane.

In accordance with aspects of illustrative embodiments, the flexible material is chosen from a hydrophobic material, and a hydrophobic silicone membrane.

In accordance with aspects of illustrative embodiments, the second housing comprises a recess formed at the sensor opening in which the sensor contact surface is disposed, and at least one of the first housing and the second housing is provided with sealing material at the corresponding one of the sensor access opening and the sensor opening, the sealing material being configured to form an enclosed chamber defined between the recess and the membrane, the sensor having a pressure sensing mechanism configured to detect enclosed air volume variation in the chamber due to movement of the membrane in response to pressure from the fluid contacting a fluid path facing side of the membrane.

In accordance with aspects of illustrative embodiments, the sensor is a contactless sensor configured to detect proximity of the membrane and determine changes in one or more membrane characteristics in response to pressure from the fluid contacting a fluid path facing side of the membrane.

In accordance with aspects of illustrative embodiments, the contactless sensor transmits at least one of light and ultrasound signals to the membrane and detects reflected signals therefrom to determine changes in one or more of the membrane characteristics.

In accordance with aspects of illustrative embodiments, at least one of the contactless sensor and the electronics are configured to define sections in a field of view of the sensor and determine which sections receive the reflected signals.

In accordance with aspects of illustrative embodiments, at least one of the contactless sensor and the electronics are configured to determine from the sections that receive the reflected signals different conditions in the fluidic module chosen from back pressure, occlusion, and at least one of a fluid pressure and flow characteristic that is out of phase from a phase of a pump provided in the fluid delivery device to control delivery of the fluid from the reservoir to the outlet.

In accordance with aspects of illustrative embodiments, the contactless sensor is chosen from an ultrasonic sensor, or a laser displacement sensor, or a Light Detection and Ranging (LiDAR) type sensor.

In accordance with aspects of illustrative embodiments, the electromechanical module further comprises a pump, a pump motor connected to the pump, and the electronics are configured to operate the pump motor to controllably deliver the fluid from the reservoir to the outlet via the pump.

In accordance with aspects of illustrative embodiments, the first housing further comprises a pump access opening and the second housing further comprises a pump opening that are at least partially aligned when the second housing is connected to first housing, at least a portion of the fluid path comprises a compressible material encasing the fluid that is accessible via the pump access opening, the pump is a peristaltic-style pump with pumping mechanism that is configured to be accessible via the pump opening to contact the compressible material of the fluid path via the pump access opening.

In accordance with aspects of illustrative embodiments, the first housing further comprises a second sensor access opening, the fluid path comprising a second sensor access area covered by a second membrane configured to be sealed relative to the fluid path to retain the fluid in the fluid path; and the second housing comprises a second sensor opening, the second sensor opening and the second sensor access opening being at least partially aligned when the second housing is connected to first housing, the electromechanical module comprising a second sensor disposed at the second sensor opening and configured to cooperate with at least part of the second membrane in the second sensor access area in the first housing to measure pressure of the fluid in the fluid path.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims.

### BRIEF DESCRIPTION OF DRAWINGS

The above and/or other aspects and advantages of the illustrative embodiments will be more readily appreciated from the following detailed description, taken in conjunction with the accompanying drawings, of which:

FIGS. 1A and 1B illustrate an example wearable FDD housing having releasably connected housing parts shown, respectively, connected together and disconnected from each other;

FIG. 2A depicts an example embodiment of a fluid delivery device having releasably connected housing parts containing a fluidic module and an electromechanical module respectively, and a pressure sensor deployed in the fluidic module that is configured to cooperate directly with the fluid in the fluid path in the fluidic module;

FIG. 2B depicts an example embodiment of a fluid delivery device having releasably connected housing parts containing a fluidic module and an electromechanical module respectively, and a pressure sensor deployed in the fluidic module that is configured to cooperate indirectly with the fluid in the fluid path in the fluidic module;

FIGS. 3 and 4 depict example embodiments of a fluid delivery device having releasably connected housing parts containing a fluidic module and an electromechanical module respectively, and a pressure sensor deployed in the electromechanical module that is configured to cooperate with the fluid in the fluid path in the fluidic module;

FIGS. 5A and 5B illustrate an example embodiment of a pressure sensor that can be employed in the fluidic module of the fluid delivery device shown in FIG. 2A;

FIG. 5C illustrates an example embodiment of a pressure sensor that can be employed in the fluidic module of the fluid delivery device shown in FIG. 2B;

FIGS. 6, 7A and 7B, and 8A and 8B illustrate example embodiments of a pressure sensor that can be employed in the electromechanical module of the fluid delivery device shown in FIG. 3 or 4, and has a pressure sensing mechanism that uses displacement of a sensor contact area;

FIGS. 9, 10 and 11 illustrate example embodiments of a pressure sensor that can be employed in the electromechanical module of the fluid delivery device shown in FIG. 3 or 4, and has a pressure sensing mechanism that uses enclosed air volume sensing;

FIG. 12 illustrate an example embodiment of a pressure sensor that can be employed in the electromechanical module of the fluid delivery device shown in FIG. 3 or 4, and has a contactless pressure sensing mechanism that uses detection of light or other energy to detect displacement or other characteristic of a sensed surface in the fluid path.

Throughout the drawing figures, like reference numbers will be understood to refer to like elements, features and structures.

### DETAILED DESCRIPTION OF ILLUSTRATIVE EMBODIMENTS

Reference will now be made in detail to illustrative embodiments, which are depicted in the accompanying drawings. The embodiments described herein exemplify, but do not limit, the illustrative embodiments by referring to the drawings.

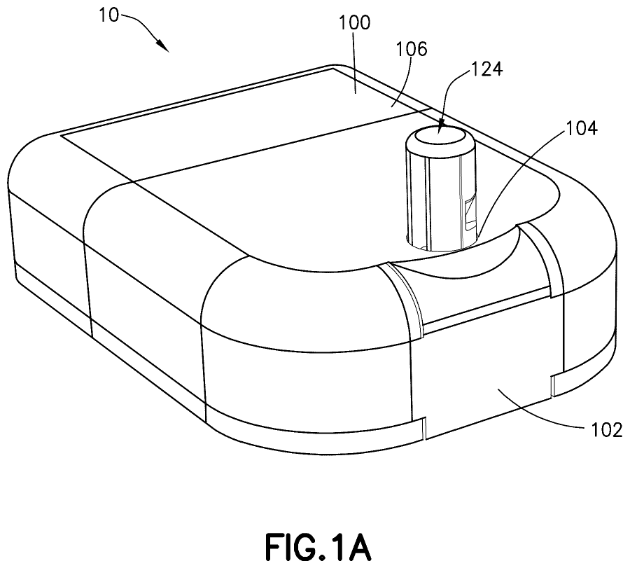

FIGS. 1A and 1B illustrate an example wearable FDD 10 having a housing 100. In accordance with example embodiments, the housing 100 can be divided into two releasably connected housing parts 102 and 106 comprising the fluidic module and the electromechanical module, respectively. FIGS. 1A and 1B illustrate a division of the housing 100 into releasably connected, side by side, housing parts 102 and 106; however, the two releasably connected housing parts 102 and 106 can be instead configured for arrangement as top and bottom releasably connected parts. The two releasably connected housing parts 102 and 106 can be equal or unequal in terms of dimensions, and can have partially overlapping portions thereof. Further, the two releasably connected housing parts 102 and 106 can employ different configurations of components and fluid pathways, and various electrical and mechanical interfaces or connections between their respective fluidic module and electromechanical module, such as described herein with respect to example embodiments shown in the drawing figures.

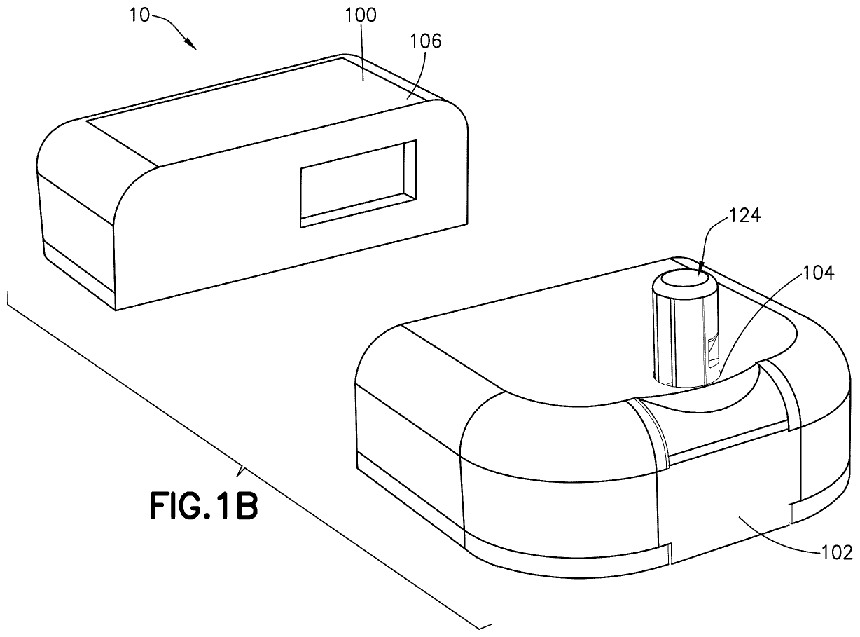

With reference to FIGS. 2A and 2B and in accordance with illustrative embodiments of the present disclosure, a wearable FDD 10 comprises two parts; that is, a fluidic module 12 and an electromechanical module 14 which can be arranged in the two releasably connected parts 102 and 106, respectively, of the housing 100. The fluidic module 12 components comprise a fluid path indicated at 16 from a fluid reservoir 18 to the patient via an outlet port 20. The outlet port 20 can be, for example, a needle in a needle hub that is fluidically connected to the fluid path 16. The fluid path 16 is separate from the electromechanical module 14 and its components (e.g., a motor 28, control electronics 26, and a power storage sub-system 30 for the FDD that can include batteries) that control the delivery of the fluid from the fluid reservoir 18 to the patient. The housing 106 can have one or more activation buttons (not shown) that when activated (e.g., depressed by a user) cause control electronics to wake-up the FDD 10 and commence a delivery mode as programmed. The programmed delivery mode can be delayed or immediate fluid discharge by pump, pulsed or continuous delivery, among other delivery modes.

In accordance with illustrative embodiments of the present disclosure, the wearable FDD 10 can have a pressure sensor 24 that is external to the fluid path 16. The control electronics 26 are programmed or otherwise configured to receive data from the pressure sensor 24 and determine fluidic pressure in at least a portion of the fluid path 16. As described below, the FDD 10 further comprises a pump 22. The pressure sensor 24 and the pump 22 can be provided in either of the fluidic module 12 and the electromechanical module 14. The pump 22 can be, for example, a peristaltic pump that is external to the fluid path 16. Alternatively, the pump 22 can be included in or used as part of the fluid path 16 such as a pump 22 as described in commonly-owned published PCT application WO 2015/157174 and incorporated herein by reference in its entirety. The reservoir 18 can be a flexible bag reservoir as described in commonly-owned published PCT application WO 2017/053284 and incorporated herein by reference in its entirety. The outlet port 20 can be a catheter and/or needle that is indicated as a needle hub 20 in FIGS. 2A-4, and that can be inserted into patient's skin by an insertion mechanism 124 shown in FIGS. 1A and 1B. The insertion mechanism can be configured, for example, as described in commonly-owned published PCT application WO 2015/164653 and incorporated herein by reference in its entirety. For example, the housing 102 can have one or more mechanical buttons, for instance the insertion mechanism 124 configured with a button to deploy a catheter and/or needle of the outlet port 20. The example embodiments in FIGS. 2A-4 have an optional fluid connector 32 configured as a fill port to the reservoir 18. An example fill port 32 is also described in the above-referenced commonly-owned published PCT application WO 2017/053284 and incorporated herein by reference in its entirety. It is to be understood that other configurations of a reservoir 18, pump 22, outlet port 20 and any associated insertion mechanism, and fill port or fluid connector 32 can be used in a FDD 10 in addition to the configurations shown and described in the present disclosure.

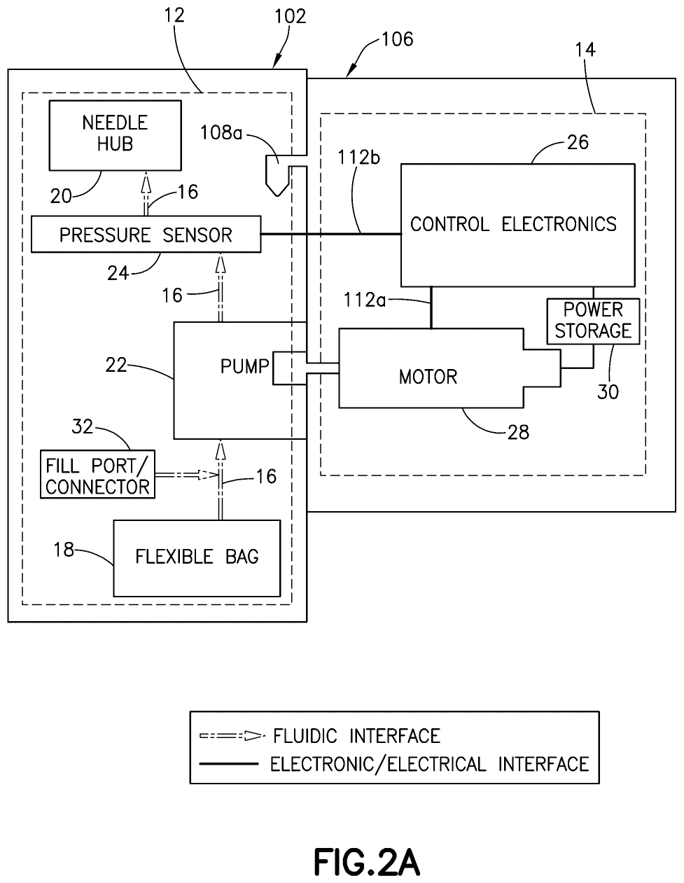

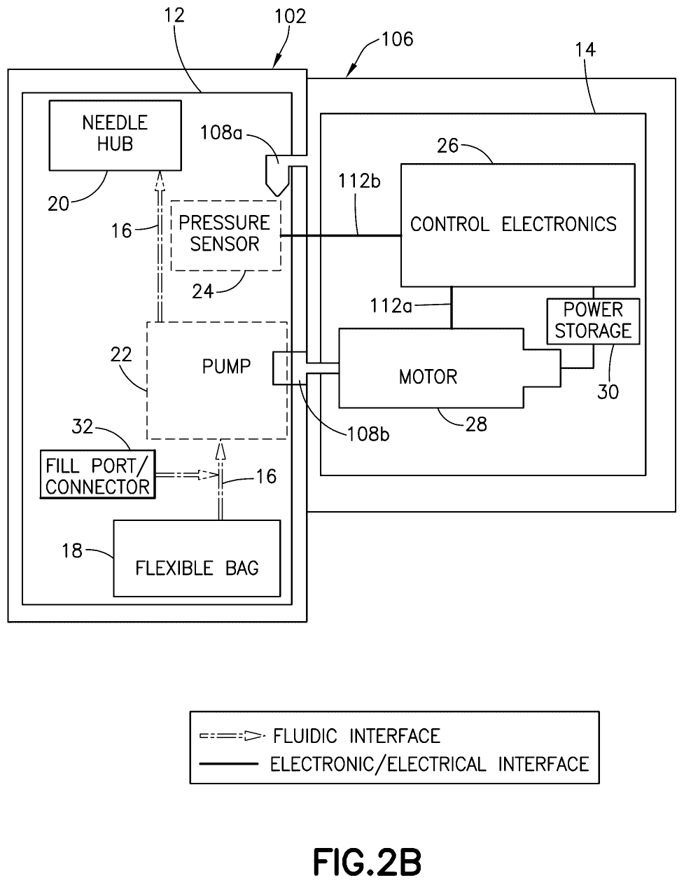

Various example embodiments of the fluidic module 12 and the electromechanical module 14 are described herein with reference to FIGS. 2A-4 and comprise different configurations of FDD 10 components. For example, the FDD 10 illustrated in FIGS. 2A and 2B comprises a fluidic module 12 and an electromechanical module 14 that are configured as a disposable single use system in accordance with an example embodiment. Other example embodiments shown in FIGS. 3 and 4 have varying arrangements of the FDD 10 components among a disposable fluidic module 12 and a reusable electromechanical module 14 in two releasably connected parts 102 and 106, respectively, of the housing 100. As described below, the example embodiments shown in FIGS. 3 and 4 are configured to transfer one or more electromechanical components from the fluidic disposable module 12 shown in FIGS. 2A and 2B to the electromechanical module 14 for cost optimization of the fluidic disposable module 12, as well as to achieve a more environmentally sustainable configuration, and to allow use of other sterilization techniques. The example embodiments described in FIGS. 3 and 4 of the present disclosure illustrate a global architecture having a disposable fluidic module and a reusable electromechanical module, as well as a pressure measurement system for pumps such as a patch pumps and other wearable pumps. The pressure measurement system described herein can be useful for large volume delivery systems (e.g., a patch pump that can deliver on the order of 50 mL of fluid medicament), as well as for smaller volume delivery systems (e.g., a patch pump that can deliver on the order of 3-9 mL of fluid medicament).

As stated above, FIG. 2A depicts an example embodiment wherein the wearable FDD 10 is configured as a disposable single use system. For example, the FDD 10 has releasably connected parts 102 and 106 of the housing 100 that each contain respectively a disposable fluidic module 12 and an electromechanical module 14, whereby the electromechanical module 14 is also disposable unlike embodiments shown in FIGS. 3-5. The releasably connected parts 102 and 106 of the housing 100 have a mechanical interface indicated generally at 108a. For example, the releasably connected parts 102 and 106 can each have hooks or other housing members that are press-fit together or otherwise frictionally engaged to retain the parts 102 and 106 of the housing 100 together (i.e., until a user intentionally separates the housing parts 102 and 106 from each other). In addition to a fluid path 16 from a fluid reservoir 18 to a patient outlet port indicated at 20, the fluidic module 12 can also comprise a pump 22 and a pressure sensor 24. The pressure sensor 24 can be in direct or indirect contact with the fluid in the fluid path 16 and described further below. The patient outlet port can be fluidically connected between the reservoir 18 and a needle hub assembly 20 that can be manually or electromechanically driven to place a needle and/or catheter into a patient's skin. The electromechanical module 14, in turn, comprises a motor 28, control electronics 26, and a power storage sub-system (e.g., batteries) 30.

With continued reference to FIG. 2A, the fluid path 16 fluidically connects the flexible bag or other type of reservoir 18 to the pump 22, and fluidically connects the pump 22 to the patient outlet 20, and includes a pressure sensor 24. An electronic/electrical interface 112b is provided between the control electronics 26 in the electromechanical module 14 and the pressure sensor 24 in the fluidic module 12, and an electronic/electrical interface 112a is provided between the control electronics 26 and the motor 28 in the electromechanical module 14. The motor 28 in the electromechanical module 14 is at least mechanically connected to the pump 22 in the fluidic module 12 as indicated at 108b (e.g., a component of the motor 22 (e.g., a rotatable shaft, or rollers in a peristaltic pump) can be releasably mechanically coupled to a pump component (e.g., a pump manifold that receives a rotatable shaft, or the exterior of a fluidic tube in the fluid path 16) for motor-pump engagement. The electronic/electrical interface 112b can be configured as press-fit electrical connectors (e.g., male and female electrical connectors, or contact pins, prong type-electrical connectors, or other form factors of releasable electrical connectors that can be provided on the respective housing parts 102 and 106 and then electrically connected when the housing parts 102 and 106 are mechanically connected together to pass current or other electrical signal between one or more components in respective ones of the fluidic module 12 and the electromechanical module 14 arranged in the housing parts 102 and 106). The arrangement of components in the fluidic module 12 and the electromechanical module 14 can be such that these two subsystems can be disposed of separately. For example, the components of the fluidic module 12 can be chosen to be mostly plastic and therefore disposed of separately from the electronic components in the electromechanical module 14.

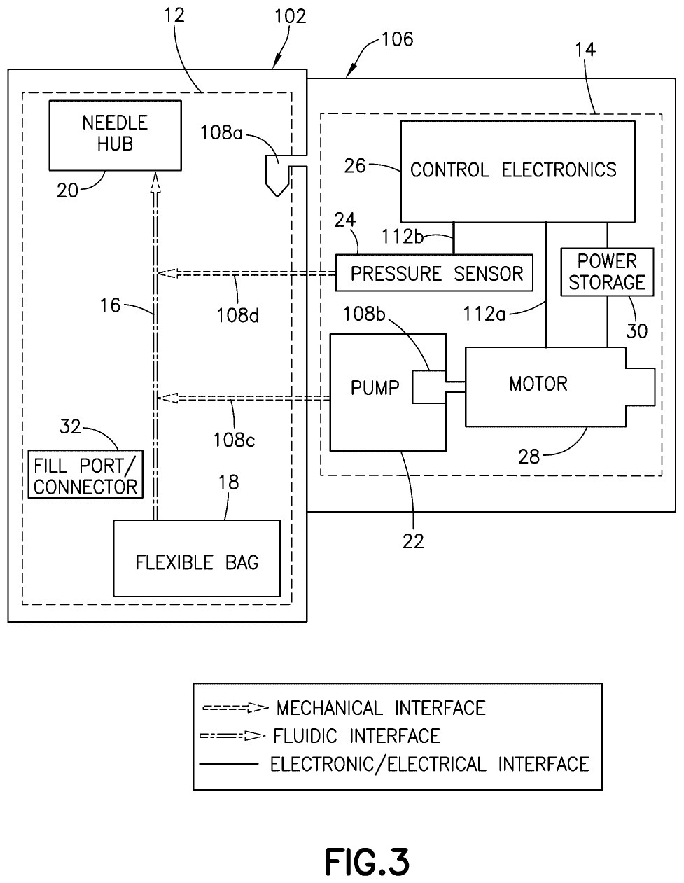

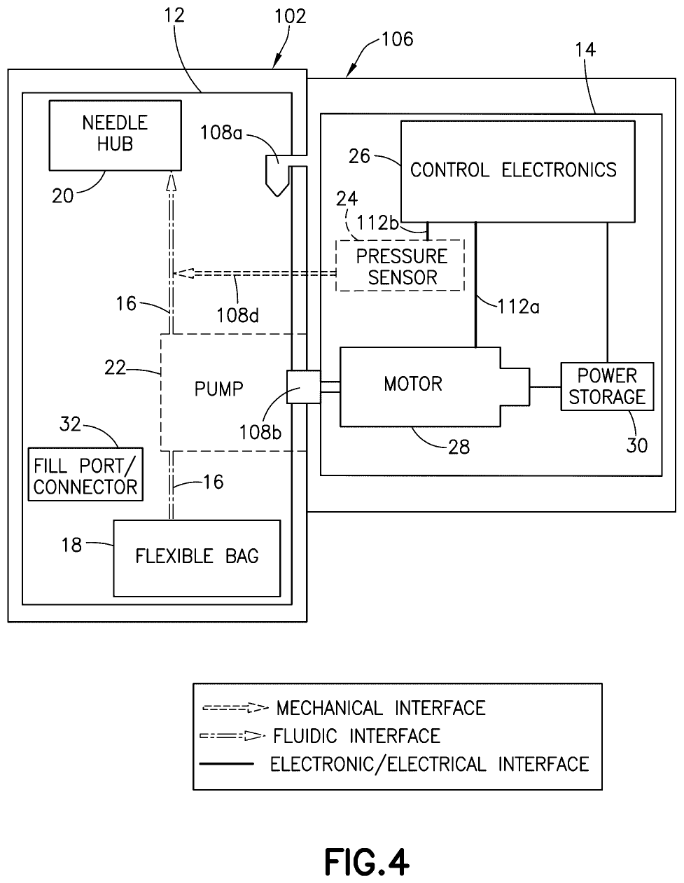

With reference to FIGS. 2A and 2B, the pressure sensor 24 of the example embodiment shown in FIG. 2A can be configured to cooperate directly with the fluid in the fluid path 16 in the fluidic module 12; that is, be in direct contact with the fluid in the fluid path 16. Examples of such pressure sensors 24 directly contacting the fluid in the fluid path 16 are described with reference to FIGS. 5A and 5B. Alternatively, the pressure sensor 24 of the example embodiment shown in FIG. 2B can be configured to cooperate indirectly with the fluid in the fluid path 16 in the fluidic module 12; that is, be in indirect contact with the fluid in the fluid path 16 via a membrane 130 as shown in FIG. 5C and operate similarly to those pressure sensors 24 disposed in the electromechanical module 14 described with reference to FIGS. 6 through 12.

The phantom lines in FIG. 2B also represents example divisions of FDD 10 components among the fluidic and electromechanical modules 12 and 14 for different reasons and to accommodate the ability to separate the respective housings 102 and 104 in which these modules 12 and 14 are disposed. For example, electronics such as the control electronics 26 and the motor 28 and the power storage system 30 can be isolated to the electromechanical module 14 for sorting separately, when disposed, from the fluidic module 12 that has primarily plastic parts. The pump 22 can also be placed in the electromechanical module 14 as illustrated in FIGS. 3 and 4 wherein the electromechanical module 14 is reusable to maximize reusable parts of the FDD 10. In either of the embodiments shown in FIGS. 2A and 2B, the interface between the pressure sensor 24 in the fluidics module 12 and the control electronics 26 in the electromechanical module 14 can simply be an electronic/electrical interface as indicated at 112b and further illustrated in FIGS. 5A and 5B. By contrast, the example embodiments described in FIGS. 3 and 4 use a mechanical interface between a pressure sensor 24 in the electromechanical module 14 and the fluid path 16 in the fluidics module 12.

FIG. 3 depicts another example embodiment wherein the wearable FDD 10 has releasably connected parts 102 and 106 of the housing 100 containing a fluidic module 12 and an electromechanical module 14, respectively. The releasably connected parts 102 and 106 of the housing 100 have a mechanical interface indicated generally at 108a similar to the interface 108a described in connection with the example embodiment of a FDD 10 shown in FIG. 2A. In contrast with the example embodiment of FIG. 2A, the embodiment of the FDD 10 shown in FIG. 3 incorporates a pump 22 and a pressure sensor 24 into the electromechanical module 14 instead of in the fluidic module 12. The electromechanical module 14 in FIG. 3 also has components to control the delivery of the fluid from the fluid reservoir to the patient such as a motor 28, control electronics 26, and a power storage sub-system 30 for the FDD 10 that can include batteries. The fluidic module 12 has a reservoir 18, optional fluid connector 32 such as a fill port fluidically connected to the reservoir 18, an outlet port 20 (e.g., at the catheter and/or needle of a needle hub of an insertion mechanism 124), and a fluid path 16 fluidically connecting the reservoir 18 and the needle hub 20. The fluid path 16 can be implemented a number of ways and can have different portions thereof made of different materials such as plastic tubing, and embedded channels in a plate, among other materials and configurations.

With continued reference to the example embodiment shown in FIG. 3, an electronic/electrical interface 112 is provided between the control electronics 26 and the motor 28 in the electromechanical module 14. The motor 28 in the electromechanical module 14 is at least mechanically connected to the pump 22 as indicated at 108b. For example, a component of the motor 22 such as a rotatable shaft, or rollers in a peristaltic pump can be releasably mechanically coupled to a pump component (e.g., a pump manifold that receives a rotatable shaft, or the exterior of a fluidic tube in the fluid path 16) for motor-pump engagement. The electromechanical module 14 has another mechanical interface 108c between the pump 22 in the electromechanical module 14 that is disposed in the housing part 106, and the fluid path 16 in the fluidic module 12 that is disposed in the housing part 102. For example, at least part of the fluid path 16 can comprise a compressible material that encases the fluid such as tubing 16 that can be selectively compressed by a pump 22 configured as a peristaltic pump with rollers and pinions that contact the exterior of the fluid path tubing 16. An example peristaltic pump is described in connection with co-pending application entitled “Peristaltic Pump for Reusable Module in Wearable Fluid Delivery Device” (Applicant ref. P-28719.US01PRO) and incorporated herein by reference in its entirety. The housing part 102 can comprise an opening wherein rollers or other tubing compressing component of the peristaltic pump 22 can externally and controllably contact the tubing 16 to selectively control movement of fluid in the fluid path 16 from the reservoir 18 to the outlet port 20.

With continued reference to the example embodiment shown in FIG. 3, a mechanical interface 108d is provided between the pressure sensor 24 in the electromechanical module 14 and the fluid path 16 (e.g., tubing or other encapsulated fluid channel configuration) in the fluidic module 12. The pressure sensor 24 is integral to, or separate from but electrically connected to, the control electronics 26 in the electromechanical module 14. The pressure sensor 24 interface 108d to the fluid path 16, and the pump 22 interface 108c to fluid path 16, can both be indirect. For example, the pump 22 can be a peristaltic pump that has indirect contact with tubing in the fluid path 16 (e.g., the housings 102 and 106 have respective and at least partially aligned openings through which the pump mechanisms can access the tubing). The pressure sensor 24 can be configured to cooperate with a membrane 130 that is part of the fluid path 16 in the fluidic module 12 and therefore be in indirect contact with the fluid path 16 fluidic module 12. Examples of such pressure sensors 24 cooperating with a membrane 130 in the fluid path 16 are described with reference to FIGS. 6 through 12.

The example embodiment shown in FIG. 3 has a fluidic module 12 that is a disposable, single use system without any electronic parts, which can be beneficial after its disposal in terms of refuse sorting at a waste management center. The electromechanical module 14 is a reusable, multiple use system by a single patient and/or multiple patients. The pressure in the fluid path 16 is measured (e.g., for detecting occlusion or other malfunction of the FDD 10) from an electronic part (e.g., a pressure sensor 24 operating with control electronics 26) in the electromechanical module 14, and is connected to the fluid path 16 in the disposable fluidic module 12 while remaining outside of the fluid path 16.

With continued reference to FIG. 3, the pump can be arranged in either module 12 and 14 such as in the fluidic module 12 and part of the fluid path 16 (e.g., the pump as described, for example, in commonly-owned published PCT application WO 2015/157174, or other type of pump disposed between the reservoir 18 and the outlet 20), or in the electromechanical module 14 as described in the above-referenced co-pending application entitled “Peristaltic Pump for Reusable Module in Wearable Fluid Delivery Device.” For example, FIG. 4 illustrates another embodiment of the FDD 10 that has releasably connected parts 102 and 106 of the housing 100 containing, respectively, a disposable fluidic module 12 and a reusable electromechanical module 14, and that is similar to the FDD 10 in FIG. 3 except for the pump 22 being arranged within the disposable fluidic module 12 while the pressure sensor 24 remains in reusable electromechanical module 14. The interface of the pump 22 to the fluid path 16 in FIG. 4 is indicated generally at 114, and can comprise an internal interface 16 as described in connection with FIG. 2A or an external interface 108c as described in connection with FIG. 3. For the example, the pump 22 in the fluidic module 12 of the FDD 10 shown in FIG. 4 can be part of the fluid path 16 as described above in connection with FIGS. 2A and 2B. Alternatively, the pump 22 in the fluidic module 12 of the FDD 10 shown in FIG. 4 can have an interface 114 comprising an external interface 108c to the fluid path 16 such as rollers and pinions of a peristaltic-type pump 22 disposed internally with respect to the fluid module 12, whereby the rollers and pinions or other components external to the fluid path 16 contact the exterior of the fluid path 16 tubing, for example. The motor 28 in the electromechanical module 14 is at least mechanically connected to the pump 22 in the fluidic module 12 as indicated at 108b. The respective parts 102 and 106 of the housing 100 can have corresponding openings or other housing features that accommodate the mechanical connection 108b of the motor 28 to the pump 22.

Different example divisions of FDD 10 components among the fluidic and electromechanical modules 12 and 14 are described herein to accommodate the ability to separate the respective housings 102 and 104 in which these modules 12 and 14 are disposed, and to achieve one or more advantages. For example, electronics such as the control electronics 26 and the motor 28 and the power storage system 30 can be isolated to the electromechanical module 14 for sorting separately, when disposed, from the fluidic module 12 that has primarily plastic parts. The pump 22 can also be placed in the electromechanical module 14 as illustrated in FIGS. 3 and 4 wherein the electromechanical module 14 is reusable to maximize reusable parts of the FDD 10. As stated previously, the different above-described example embodiments of FDDs 10 separate a fluidics module 12 from an electromechanical module 14 to avoid or at least minimize electronics in the fluidics module 12. Also, the above-described example embodiments of FDDs 10 in FIGS. 3 and 4 externalize a pressure sensor 24 (e.g., a fluid path 16 detection area is accessible from outside of the fluidics module 12) to allow detection of pressure differential such as detection of an occlusion between the reservoir 18 and the outlet 20 in the fluidics module 12 from a pressure sensor 24 disposed in the electromechanical module 14. In addition, the example embodiments of FDDs 10 of the present disclosure allow for more options for sterilization. For example, certain sterilization methods such as gamma or X-ray radiation methods can be used to sterilize the fluidics module 12 when there are no electronics within the fluidics module 12 that can be harmed by gamma or X-ray radiation. A FDD 10 is more sustainable when it uses only mechanical parts in the fluidics module 12 (e.g., the FDD 10 in FIG. 3 or 4) and gamma or X-ray sterilization thereof. The reusable electromechanical module 14 can be packaged separately as cartridges and does not need to be sterilized at all. In addition, the more components of the FDD 10 that are disposed in the reusable electromechanical module 14, the more optimized that FDD 10 is for minimizing direct costs of producing the FDD 10 (i.e., Cost of Goods Sold (COGS)). If the electromechanical module 14 employs a pressure sensor 24 having an indirect fluid contact configuration, then sterilization of the electromechanical module 14 can be avoided by providing a cap or septum outlet or other component to the sensor opening 156 (FIG. 5C) on the electromechanical module 14, for example, or otherwise incorporated therein to guarantee the sterility of the pressure sensor sub-part 24 that is in indirect contact with the fluid via the sensor access area 138 in the fluidic module 12. If this component itself is sterile before assembly, then the entire electromechanical module 14 does not need to be sterilized.

It is to be understood that FDD 10 is generally for infusion and can use various delivery modes (e.g., pulsed or continuous delivery), and can employ any of these various modes for delivery over a relatively short period of time, or over longer periods of time. Further, the FDD 10 can be employed to deliver various medicaments (e.g., various drugs and/or other various medical fluids) for various medical fluid delivery applications. Example pumps 22 are described herein, but it should be understood that other types of pumps can be used and various delivery methods can be used by the example embodiments such as, but not limited to, the above-reference delivery modes.

Reference is now made to FIGS. 5A through 12, which illustrate different types of pressure sensors 24 that can be employed in a FDD 10 in accordance with various example embodiments. For clarity, FIGS. 5A through 12 depict only some of the components in the fluidic module 12 and electromechanical module 14 of an example FDD 10 and only a portion of their respective housings parts 102 and 106. Further, only a portion of the fluid path 16 is shown in FIGS. 5A through 12, and the fluid path 16 portion is shown with example fluid 34 therein. The fluid path 16 has a casing 126 or frame or other structure to define a sensor access area 138 in the fluid path 16 (e.g., via a raised portion 136 extending from a main surface of the casing 126) that can be integral to the fluid path 16 as illustrated in FIGS. 5A and 5B, or separate from but fluidically connected to the fluid path 16. For example, at least part of the fluid path 16 can comprise a portion 134 configured as a plate with one or more fluid channels formed therein as described in commonly owned WO 2016/048878. Further, the casing 126 can be a raised portion 136 on a plate or planar surface that surrounds an exposed portion of a fluid channel embedded in the plate to define a sensor access area 138 such as a cavity as described in commonly owned WO 2016/090265. The contact surface 128 of the pressure sensor 24 and/or the membrane 130 can be seated in the cavity 138 and fluidically sealed with respect to the casing 126 and therefore the fluid path 16. Alternatively, the portion 134 is part of the casing 126 and the fluid path 16 is fluidically connected to the portion 134 to allow fluid from the fluid path 16 to flow therethrough. Alternatively, the fluid path 16 can comprise tubing, and the portion 134 is part of the casing 126 and is fluidically sealed on opposite ends thereof to the tubing in the fluid path 16. A sensing mechanism 132 is shown in the pressure sensor 24, and different example sensing mechanisms 132 are described below.

FIGS. 5A and 5B illustrate a first example embodiment of a pressure sensor 24, which is disposed in a fluidic module 12 and is in direct contact with fluid 34 in the fluid path 16 as described above in connection with FIG. 2A. The electronic/electrical interface 112b between the pressure sensor 24 in the fluidic module 12 and the control electronics 26 in the electromechanical module 14 is shown as electrically connected and disconnected in FIGS. 5A and 5B, respectively. FIG. 5C illustrates a second example embodiment of a pressure sensor 24, which is disposed in a fluidic module 12 but instead indirectly contacts the fluid 34 in the fluid path 16 via a membrane 130 as described above in connection with FIG. 2B.

With continued reference to FIGS. 5A, 5B and 5C, the pressure sensor 24 is sealed in a casing 126, or casing 126/path portion 134 combination, in which the fluid in the fluid path 16 needs to transit or flow through to measure fluid pressure in the fluid path 16. The electrical interface between the pressure sensor 34 in the fluidics module 12 and electronics (e.g., printed circuit board (PCB)) comprising the control electronics 26 in the electromechanical module 14 can be via cooperating contact pins and sockets disposed, for example, in the respective modules 12, 14 and their corresponding housings 102, 106. In accordance with an example embodiment, the fluid path 16 can have an exposed portion or access area thereof indicated generally at 138, and the housing 102 comprising the fluidics module 12 can be provided with at least one sensor access opening 152. The housing 106 comprising the electromechanical module 14 can be provided with at least one sensor opening 156 arranged therein such that the sensor opening 156 and the sensor access opening 152 are at least partially aligned when the housing 106 is connected to the housing 102, and data from a sensor having direct or indirect contact with the fluid in the sensor access area 138 can be provided to the controller 26 in the electromechanical module 14.

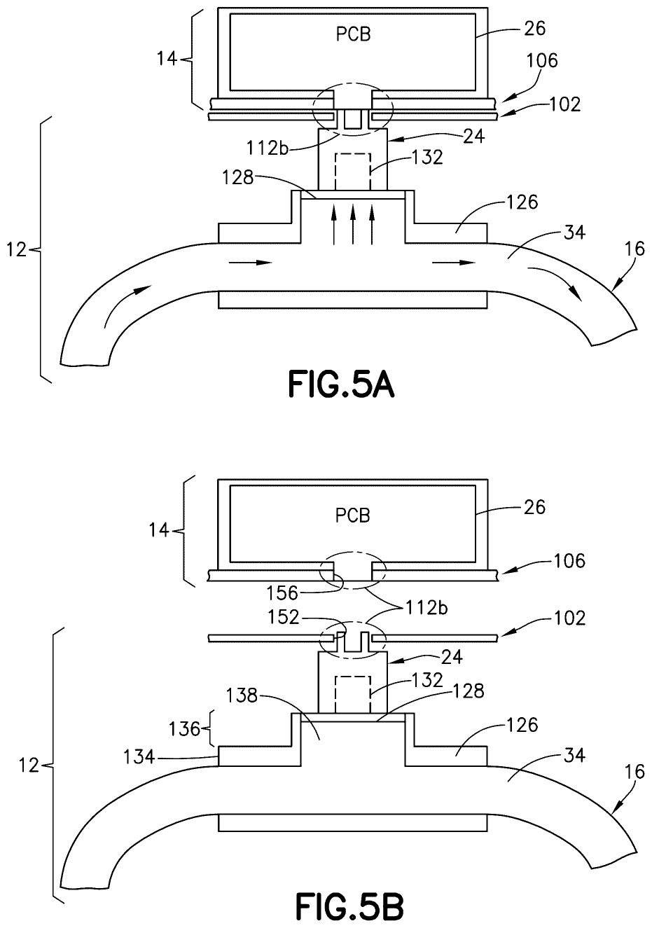

With reference to FIG. 6 and in accordance with another example embodiment, the pressure sensor 24 is disposed in the electromechanical module 14 and indirectly contacts fluid in the fluid path 16 of the fluidic module 12 as described above in connection with FIGS. 3 and 4. For clarity, the partial housings 102 and 106 enclosing respective ones of the fluidic module 12 and the electromechanical module 14 are shown as disconnected. The housing 102 containing the fluidic module 12 has a portion thereof comprising a mechanical interface 108d with respect to the housing 106. More specifically, the mechanical interface 108d is provided between the pressure sensor 24 in the electromechanical module 14 and the fluid path 16 (e.g., tubing or other encapsulated fluid channel configuration) in the fluidic module 12. For example, the pressure sensor 24 can be configured to cooperate with a membrane 130 in the mechanical interface 108d that is part of the fluid path 16 in the fluidic module 12 such that the pressure sensor 24 indirectly contacts with the fluid path 16 fluidic module 12. The fluid path 16 can have an exposed portion or access area thereof indicated generally at 138 that is fluidically sealed with respect to the membrane 130. Examples of different pressure sensors 24 and membranes 130 in the fluid path 16 will now be described with reference to FIGS. 7A through 12.

FIGS. 7A and 7B illustrate an example embodiment of a pressure sensor 24 that is external to the fluid path 16 in the fluidics module 12, and cooperates with an intermediate deformable surface in the mechanical interface 108d that is disposed between the housings 102 and 106 and their corresponding ones of the fluidic module 12 and the electromechanical module 14. For example, a membrane 130 is chosen as the intermediate deformable surface wherein the membrane 130 is flexible and the pressure sensor 24 is configured to detect a displacement of the membrane or force of the membrane 130 against the sensor contact surface 128 when fluid 34 pressure increases in the sensor access area 138 and exceeds a designated threshold for an occlusion 140 or other condition for which the pressure sensor 24 is employed to detect in the FDD 10. The membrane 130 can be a diaphragm of flexible material that can deform when pressure from fluid 34 in the fluid path 16 exceeds this designated threshold. The membrane 130 can be a hydrophobic material such as a hydrophobic silicone membrane, for example. The sensor 24 can be configured to either measure a deformation or deflection of the membrane 130 such as a membrane displacement length and/or a pressure such as a force of the membrane 130 applied to the sensor contact surface 128.

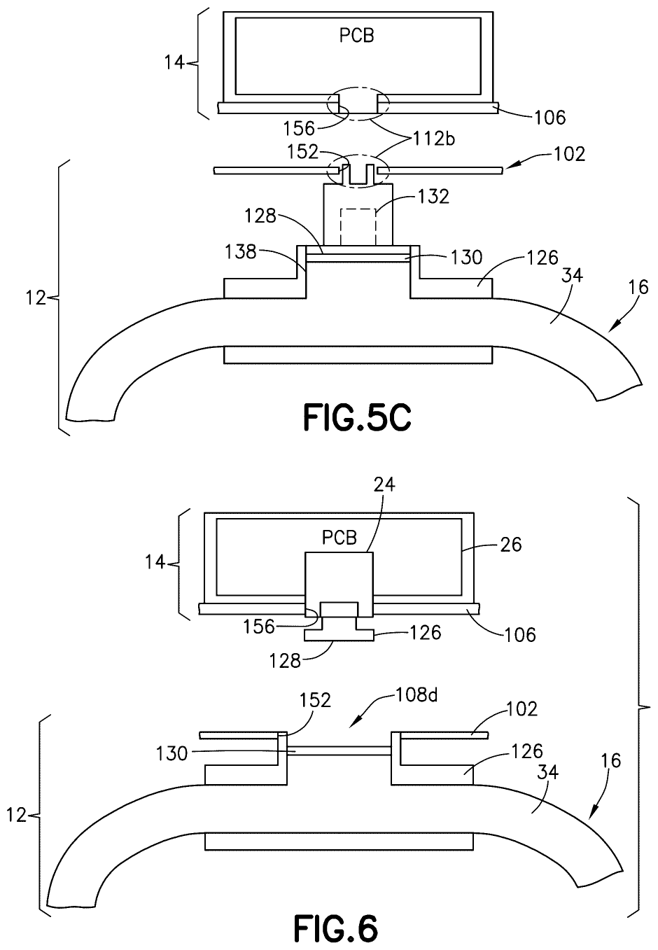

In accordance with another example embodiment shown in FIGS. 8A and 8B, the membrane 130 of the mechanical interface 108d is rigid, instead of flexible as in the example embodiment shown in FIGS. 7A and 7B. For example, the sensor access area 138 can be delineated by raised portion or side wall(s) 136 that slidably retain the rigid membrane 130 for a fluidic seal with respect to the fluid 34 in the fluid path 16 yet allow for translation of the rigid membrane 130 along the side wall(s) 136 when fluid 34 applies a designated force on the membrane 130 that is sufficient to move it and that exceeds a designated force threshold indicative of an occlusion 140 or other condition for which the pressure sensor 24 is employed to detect in the FDD 10. For example, the membrane 130 can be configured similarly to a stopper in a syringe whereby the membrane 130 forms a fluidic seal with respect to the sensor access area 138 in the fluid path 16 yet its displacement resulting from fluid 34 pressure in the fluid path 16 can be measured.

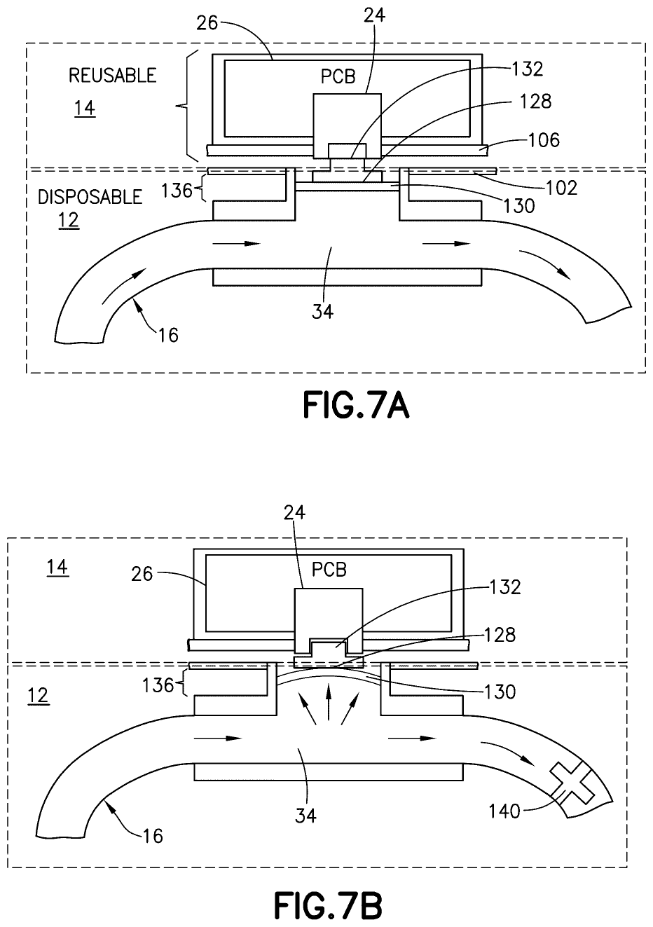

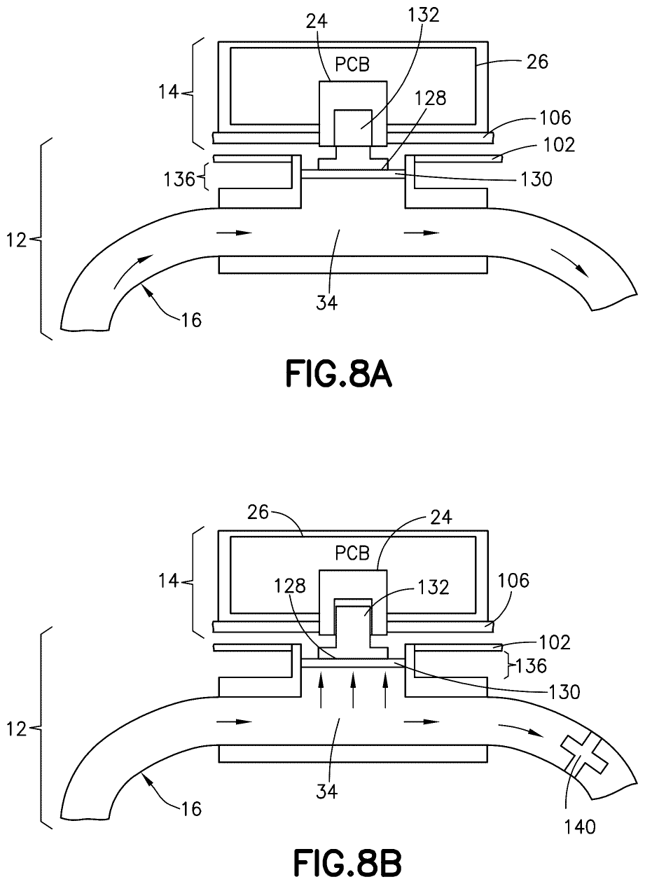

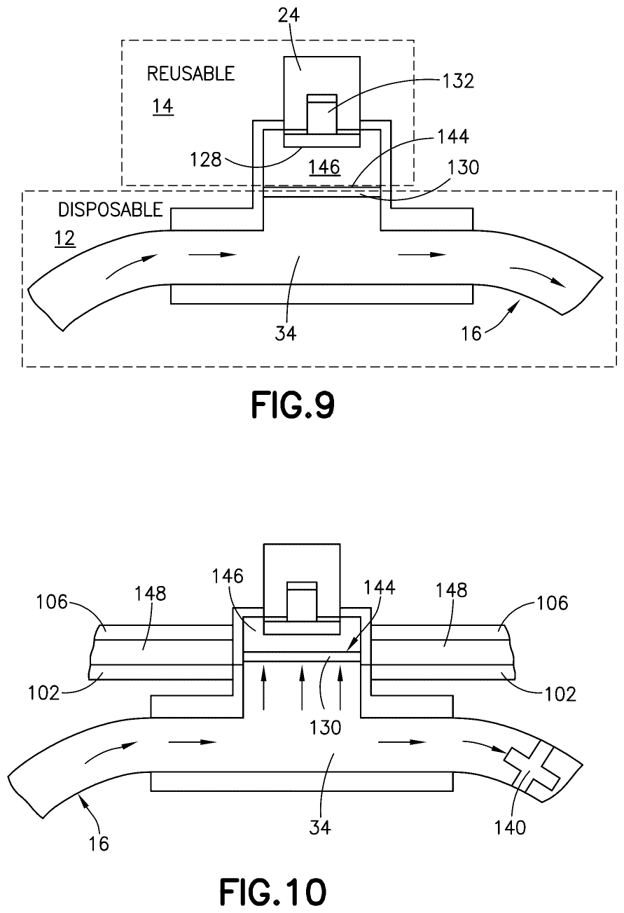

In the example embodiments described with reference to FIGS. 7A and 7B and FIGS. 8A and 8B, the sensor contact surface 128 abuts the membrane 130. In accordance with another example embodiment shown in FIGS. 9, 10 and 11, the sensor contact surface 128 of the sensor 24 does not abut the membrane 130. Instead, the sensor 24 can be configured to comprise a sensing area 144 that is entirely or at least partially in the electromechanical module 14. The housings 102, 106 can be provided with gaskets or other sealing materials 148 that abut each other when the housings 102 and 106 of the FDD 10 are interconnected to form a sealed chamber of air between the sensor contact surface 128 and the membrane 130. For example, the gasket(s) 148 on one or both of the housings 102 and 106 at the corresponding locations of the sensor access area 138 of the fluid module 12 and the sensing area 144 of the electromechanical module 14 can compress with respect to the other housing to form a sealed chamber 146 wherein pressure differentials created by displacement of the membrane 130 can be measured by the sensor to determine if pressure exceeds a designated threshold indicative of an occlusion 140 or other condition for which the pressure sensor 24 is employed to detect in the FDD 10. As illustrated in FIGS. 10 and 11, respectively, the membrane 130 can be rigid or flexible. In either case, displacement of the membrane 130 can be into the sensing area 144 of the electromechanical module 14, for example. In the case of a rigid membrane 130 as shown in FIG. 10, side wall(s) that define the sensing area 144 can be used to guide translation of the rigid membrane 130 therein in a manner similar to the side wall(s) 142 in the embodiment shown in FIGS. 8A and 8B.

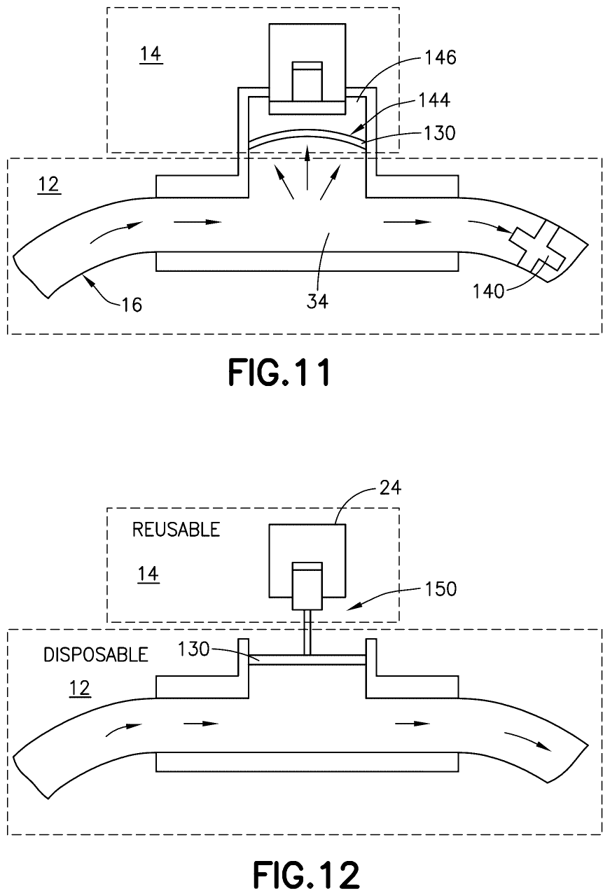

In accordance with another example embodiment shown in FIG. 12, the sensor 24 employs a contactless-type sensor instead of a sensor contact surface 128 and physical pressure force detection, and thereby is able to removes all physical connection between a membrane 130 present on the fluidic path 16 and the sensor 24 in the electromechanical module 14. For example, the fluidic module 12 can have a movable membrane (e.g., the various rigid and flexible membranes 130 described above in connection with FIG. 7A though 11) that is able to move and/or change shape depending on the fluid path 16 pressure. The electromechanical module 14 can have a contactless sensor 24 able to read and/or quantify the membrane 130's movement(s) such as can be accomplished with a proximity sensor, for example. The sensor 24 can be, for example, a pair of IR emitter/receptor arranged respectively between a sensor 24 in an electromechanical module 14 and a corresponding sensor access area 138 in a fluidic module 12. The sensor 24 can be, for example, an ultrasonic sensor, or a laser displacement sensor, or a Light Detection and Ranging (LiDAR) type sensor. An advantage of a contactless-type sensor 24 is tolerance of positioning. In other words, a distance calibration can be performed when inserting or otherwise attaching a new fluidic cartridge 102 (i.e., new fluidic module 12 in its corresponding housing 102) to an FDD 10's reusable housing 106 with an electromechanical module 14. Such a distance calibration can ensure that essentially the same measurement accuracy is achieved for all replaceable fluidic cartridges 102 even if positioning is not the same. For example, a software-based auto adjustment system can be provided to the software of a controller in the control electronics 26 that detects when the reusable housing 16 is reattached to a new fluidic cartridge 102 (e.g., using outputs from electrical contacts provided at an electrical interface 112 disposed between the housings 102 and 106 for the purpose of detecting housing connection or for another other purpose (e.g., to operate an sensor 24 and/or pump 22 when the housings are connected.

With continued reference to the example embodiment shown in FIG. 12, using an optical-type sensor 24 can also provide additional value if a quadrant-based sensor (or similar technology) is used to define plural sections in the field of view of the sensor. For example, depending on where the reflected light from the membrane 130 hits the sensor 24 (e.g., which quadrant), the control electronics 26 can be programmed to determine measurements and other derived information related to back pressure, occlusion, or a fluid pressure or flow characteristic that is out of phase from the pump 22 phase. As such, the signal processing of the control electronics 26 can be configured to increase the value or relevance of information gathered from light or ultrasound or other energy 150 transmitted to the membrane 130 from the sensor 24 and reflected back to the sensor 24 from the membrane 130 to include at least displacement length and speed of the membrane as well as pattern of deformity of the membrane 130 by fluid pressure. For example, the pattern can identify two-dimensional offset and/or three-dimensional tilting of the membrane, which can be useful for the above-described distance or placement calibration for consistency of pressure measurements using the sensor 24 in a reusable module 106 with different fluidic modules 102 and therefore compatibility of the reusable module 106 with different replaceable fluidic modules 102.

It is to be understood that the fluidic module 12 and the electromechanical module 14 can be disposed in respective, connectable housings 102 and 106, or can both be disposed in a single integral housing 100. In either housing configuration, the sensor 24 can be deployed as a separate component that is external to the fluid path 16. As described herein, different embodiments of the sensor 24 allow the sensor to be deployed in an electromechanical module 14 in a housing part 106 that is separate from a housing part 102 containing the fluid path 16, thereby making separation of the fluidic module 12 from the electromechanical module 14 easier.

It is also to be understood that the fluidic module 12 and the electromechanical module 14 can each have more than one corresponding sets of sensor interfaces in their housings 102 and 106 (e.g., plural mechanical interfaces 108d between respective sensors 24 and corresponding sensor access areas 138) to accommodate a plurality of sensors 24 located at different points along the fluid path 16. For example, a plurality of infusion sets can be connected to the FDD 10 and a sensor 24 can be provided per infusion set (e.g., the FDD 10 is configured as a wearable patch on the body with one or more infusion sets connected by tubes to the pump to treat certain diseases such as Parkinson's disease using several infusion sites at the same time. As another example, for a single direction pump used also for filling the reservoir 18 as well as for reversing direction and controlling discharge therefrom to the outlet 20, the respective housings 102 and 106 can have a first sensor interface 108d near the location of the reservoir 18 for pressure sensing during a filling operation and a second sensor interface 108d closer to the outlet 20 for detecting an occlusion in the fluid path 16 or other malfunction of the FDD 10 (e.g., incorrect placement of a needle/catheter in the skin of a patient).

As stated above, by moving a sensor 24 into an electromechanical module 14, the electromechanical module 14 can be configured to be reusable with replacement and disposal of the fluidic module 12 only, which would make the FDD 10 much more environmentally sustainable. Such a configuration for an FDD 10 also realizes advantages in terms of expanded options for means of sterilization such as relying less on ethylene oxide (EtO) sterilization and using instead Gamma sterilization, for example, which is incompatible with the electronic components found in some wearable FDD configurations.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. Also, the drawings are representative of example components in the illustrative embodiments and these representations can have dimensional scale different from that shown in the FIGS. 1-12.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, an artificial intelligence (Al) chip, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. An AI chip can be used, for example, to perform signal processing of data from a sensor(s) 24 and adapt an injection profile performed by the FDD 10 and/or predict risk from occlusion or device defect events, among other functions.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., erasable programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof. Also, either of the fluidic module 12 or the electromechanical module 14 can be provided with a communication circuit and/or antenna or RFID tag as needed to obtain data from a sensor 24 or control electronics 26 or other data source about the module 12,14 (e.g., a device identifier or software version or manufacturer information that is read from an RFID tag) and communicate that data to another device via WiFi, BlueTooth, cellular communication, wide area network, and so on).

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A fluid delivery device comprising:
a first housing comprising a fluidics module and a sensor access opening, the fluidics module comprising a reservoir of fluid and a fluid path that fluidically connects the reservoir to an outlet configured to output the fluid from the fluid path to a patient, the fluid path comprising a sensor access area covered by a membrane configured to be sealed relative to the fluid path to retain the fluid in the fluid path; and
a second housing separate from the first housing and comprising an electromechanical module and a sensor opening, the sensor opening and the sensor access opening being at least partially aligned when the second housing is connected to first housing, the electromechanical module comprising a sensor and electronics configured to process data output from the sensor, the sensor comprising a sensor contact surface disposed at the sensor opening of the second housing and configured to cooperate with at least part of the membrane in the sensor access area in the first housing to measure pressure of the fluid in the fluid path.

2. The fluid delivery device of claim 1, wherein the sensor access area is formed by a casing fluidically connected to the fluid path and having side walls that define the sensor access area, and the membrane is a rigid material and is operable to be displaced along the side walls in response to pressure from the fluid contacting a fluid path facing side of the membrane, the sensor having a pressure sensing mechanism configured to detect displacement of the sensor contact surface when the sensor contact surface is in contact with the membrane and moves due to displacement of the membrane.

3. The fluid delivery device of claim 1, wherein membrane is a flexible material and is deformable in response to pressure from the fluid contacting a fluid path facing side of the membrane, the sensor having a pressure sensing mechanism configured to detect movement of the sensor contact surface when the sensor contact surface is in contact with the membrane and moves due to deformation of the membrane.

4. The fluid delivery device of claim 3, wherein the flexible material is chosen from a hydrophobic material, and a hydrophobic silicone membrane.

5. The fluid delivery device of claim 1, wherein the second housing comprises a recess formed at the sensor opening in which the sensor contact surface is disposed, and at least one of the first housing and the second housing is provided with sealing material at the corresponding one of the sensor access opening and the sensor opening, the sealing material being configured to form an enclosed chamber defined between the recess and the membrane, the sensor having a pressure sensing mechanism configured to detect enclosed air volume variation in the chamber due to movement of the membrane in response to pressure from the fluid contacting a fluid path facing side of the membrane.

6. The fluid delivery device of claim 1, wherein the sensor is a contactless sensor configured to detect proximity of the membrane and determine changes in one or more membrane characteristics in response to pressure from the fluid contacting a fluid path facing side of the membrane.

7. The fluid delivery device of claim 6, wherein the contactless sensor transmits at least one of light and ultrasound signals to the membrane and detects reflected signals therefrom to determine changes in one or more of the membrane characteristics.

8. The fluid delivery device of claim 7, wherein at least one of the contactless sensor and the electronics are configured to define sections in a field of view of the sensor and determine which sections receive the reflected signals.

9. The fluid delivery device of claim 8, wherein at least one of the contactless sensor and the electronics are configured to determine from the sections that receive the reflected signals different conditions in the fluidic module chosen from back pressure, occlusion, and at least one of a fluid pressure and flow characteristic that is out of phase from a phase of a pump provided in the fluid delivery device to control delivery of the fluid from the reservoir to the outlet.

10. The fluid delivery device of claim 6, wherein the contactless sensor is chosen from an ultrasonic sensor, or a laser displacement sensor, or a Light Detection and Ranging (LiDAR) type sensor.

11. The fluid delivery device of claim 1, wherein the electromechanical module further comprises a pump, a pump motor connected to the pump, and the electronics are configured to operate the pump motor to controllably deliver the fluid from the reservoir to the outlet via the pump.

12. The fluid delivery device of claim 11, wherein the first housing further comprises a pump access opening and the second housing further comprises a pump opening that are at least partially aligned when the second housing is connected to first housing, at least a portion of the fluid path comprises a compressible material encasing the fluid that is accessible via the pump access opening, the pump is a peristaltic-style pump with pumping mechanism that is configured to be accessible via the pump opening to contact the compressible material of the fluid path via the pump access opening.

13. The fluid delivery device of claim 1, wherein the first housing further comprises a second sensor access opening, the fluid path comprising a second sensor access area covered by a second membrane configured to be sealed relative to the fluid path to retain the fluid in the fluid path; and
the second housing comprises a second sensor opening, the second sensor opening and the second sensor access opening being at least partially aligned when the second housing is connected to first housing, the electromechanical module comprising a second sensor disposed at the second sensor opening and configured to cooperate with at least part of the second membrane in the second sensor access area in the first housing to measure pressure of the fluid in the fluid path.
